(19) United States
(12) Patent Application Publication
(10) Pub. No.: US 2018/0110737 A1
(43) Pub. Date: Apr. 26, 2018

(54) EXTENDED-RELEASE ORAL PHARMACEUTICAL COMPOSITION OF AMPHETAMINE

(71) Applicant: Gavis Pharmaceuticals, Somerset, NJ (US)

(72) Inventors: James GAREGNANI, Somerset, NJ (US); Paulvia Samuel Robert KENNEDY, Somerset, NJ (US)

(21) Appl. No.: 15/789,745

(22) Filed: Oct. 20, 2017

Related U.S. Application Data
(60) Provisional application No. 62/412,490, filed on Oct. 25, 2016.

(51) Int. Cl. A61K 9/50; A61K 9/00; A61K 31/137

(52) U.S. Cl. CPC A61K 9/5026; A61K 31/137; A61K 9/0053

(57) ABSTRACT

Extended-release oral pharmaceutical compositions of amphetamine, a pharmaceutically acceptable salt, enantiomer, or combination thereof are provided. The compositions comprise drug-cation exchange resin complex particles which comprise extended-release coated amphetamine-cation exchange resin-matrix particles coated with an extended-release coating. Both the matrix and the extended-release coating comprise the same polymer or copolymer, and preferably the composition is devoid of uncoated amphetamine-ion exchange resin complex particles and amphetamine particles which are not complexed with an ion exchange resin.

This application claims priority to U.S. Provisional patent Application No. 62/412,490, filed 25 Oct. 2016. To the extend appropriate, a claim of priority is made to the above disclosed application.

### BACKGROUND OF THE INVENTION

### (a) Field of the Invention

The present invention is directed to an extended-release oral pharmaceutical composition comprising amphetamine, a pharmaceutically acceptable salt, enantiomer, or combination thereof. The invention is further directed to use of said composition for treating Attention Deficit Hyperactivity Disorder (ADHD).

### (b) Description of the Related Art

Use of ion-exchange resins to form a drug-ion exchange resin complex is well known in the art. For example, U.S. Pat. No. 2,990,332 describes use of an ion-exchange resin to form a complex with ionic drugs and thereby delay the drug release from such complexes. Such delay in drug release was deemed to be of relatively short duration.

U.S. Pat. Nos. 3,138,525; 3,499,960; 3,594,470; Belgian Pat. No. 729,827; German Pat. No. 2,246,037 describe use of ion-exchange resin complexes with water-permeable diffusion barrier coatings to alter the release of drugs from the drug-ion exchange resin complex.

U.S. Pat. Nos. 4,221,778 and 4,847,077 disclose treatment of drug-ion exchange resin complexes with water soluble, hydrophilic impregnating (solvating) agents such as polyethylene glycol and others so as to enable the coating of drug-ion exchange resin complexes with a water-permeable diffusion barrier. These patents indicate that the drug-ion exchange resin tended to swell when in contact with water, causing the coating layer to fracture and prematurely release the drug thereby adversely impacting the purpose of the coating (i.e., control release). European Patent No. EP 0,171,528 B1 discloses a similar resin treatment using glycerine to improve coatability.

European Patent Nos. EP 0,254,811 B1 and EP 0,254,822 B1, respectively, disclose use of hydroxypropylmethylcellulose, hydroxypropyl cellulose, hydroxypropyl sorbitol, sorbitol, and polyvinylpyrrolidone as impregnating agent in amount up to about 20 percent by weight based on the weight of resin and use of high molecular weight polymers to improve coatability.

U.S. Pat. No. 4,996,047 discloses use of a drug content above a specified value in the drug-ion exchange resin complex to avoid the swelling of the drug-ion exchange resin complex and thereby minimizing the rupture of the coating.

U.S. Pat. No. 5,368,852 discloses that despite the use of impregnating agents, certain preservatives used in the liquid preparation tend to cause the rupture of the diffusion barrier coating of the drug-ion exchange resin complex. The patent suggests use of a preservative that does not cause the rupture of the coating membrane.

Sustained or prolonged release dosage forms of various drugs are known and commercially available. However, there are only a few products available that provide sustained release of the drug from the very fine particles of coated drug-ion exchange complexes. U.S. Application Pub. No. US 2005/0181050, mentions that few modified release liquids containing drug-loaded ion exchange resin particles are commercially available. It further states that such products require several time consuming steps and require the use of a potentially hazardous step of coating from a solvent based solution. The regulatory authorities require that such solvents are thoroughly removed from the pharmaceutical products before ingestion.

U.S. Pat. No. 6,001,392 describes certain acrylate based (e.g., EUDRAGIT polymer system) and ethyl cellulose (e.g., SURELEASE, AQUACOAT) polymers for coating a drug ion exchange resin complex using either a solvent or aqueous based coating to achieve sustained release of the drug from the drug-ion exchange resin complex. The sustained-release composition disclosed in the patent contains a mixture of coated and non-coated drug-ion exchange resin complex particles.

U.S. Application Pub. No. US 2003/0099711 discloses use of an ethyl cellulose polymer in an aqueous based barrier coating system. This publication further discloses use of an enteric coating as an optional added coating to delay the drug release and use of several solvating agents for impregnation with drug-resin particles including those disclosed in U.S. Pat. No. 4,221,778 and propylene glycol, mannitol, lactose, methylcellulose, hydroxypropylmethylcellulose, sorbitol, polyvinylpyrrolidone, carboxypolymethylene, xanthan gum, propylene glycol alginate or combinations thereof.

U.S. Pat. No. 5,186,930 discloses stable sustained release pharmaceutical compositions comprising a drug-resin complex suspended in a liquid carrier for oral administration. The drug-resin complex comprises a drug-resin particle coated with a first inner coating of a wax and a second outer coating of a polymer.

U.S. Application Pub. No. 2005/0181050 discloses a drug formulation comprising drug-ion exchange resin complex particles without an impregnating agent and coated with extended release coatings.

Extended-release aqueous suspension of amphetamine has been approved recently under the brand name Dyanavel® to Tris Pharma (Jew Jersey, USA) for the treatment of ADHD. The suspension contains a mixture of barrier coated and uncoated amphetamine-ion exchange resin complex particles. The barrier coat particles contain polyvinylpyrrolidone as an impregnating agent for drug-resin complex particles and polyvinyl acetate in the barrier coating.

U.S. Pat. Nos. 8,062,667; 8,597,684; 8,997,684 and U.S. Application Pub. No. 2007/0215511 disclose pharmaceutical compositions of drug-ion exchange resin complex admixed with a release retardant water-insoluble polymer which is further coated with a highly flexible, substantially tack-free, non-ionic, water-insoluble, water permeable, aqueous based diffusion membrane to provide controllable modified release of the drug in the gastrointestinal tract (GIT) for a duration of up to about 24 hours. These patents and publication mentions issue of tackiness associated with acrylate and methacrylate based aqueous dispersion coating systems for coating a drug ion exchange resin complex. Further, the patents and publication mentions that use of water soluble impregnating (solvating) agents is not necessary to achieve the prolonged release of drug.

There still exists a need of a novel and simple extended-release composition of amphetamine in the form of coated drug-ion exchange resin complex particles which can be manufactured by a cost effective process.

### SUMMARY OF THE INVENTION

The present invention provides the following aspects, subject-matters and preferred embodiments, which respectively taken alone or in combination, further contribute to solving the object of the present invention.

An improved extended-release pharmaceutical composition of amphetamine has been developed. The composition is made by complexing amphetamine or a pharmaceutically acceptable salt, enantiomer, or combination thereof with an ion-exchange resin to provide a drug-ion exchange resin complex, forming a matrix of such complex with acrylate copolymer, and coating of such matrix particles with a composition comprising the same acrylate copolymer. The coated drug-ion exchange resin complex-matrix particles provide extended release of amphetamine in the gastrointestinal tract for a duration of up to about 24 hours.

In one aspect, the present invention provides an oral pharmaceutical composition comprising an extended-release coating over drug-cation exchange resin complex particles which comprise amphetamine, a pharmaceutically acceptable salt, enantiomer or a combination thereof bound to a cation exchange resin, wherein—
(a) said extended-release coated drug-cation exchange resin complex particles comprise ethyl acrylate and methyl methacrylate copolymer in a matrix with the drug-cation exchange resin complex, and
(b) the extended-release coating over the drug-cation exchange resin complex-matrix particles comprise ethyl acrylate and methyl methacrylate copolymer.

The cation exchange resin is preferably a sulfonated copolymer of a polystyrene crosslinked with divinylbenzyl. The drug-cation exchange resin complex particles have a particle size preferably in the range of about 40 to about 250 microns.

In a further aspect, the composition comprises a mixture of d- and I-enantiomers of amphetamine. In another aspect, d- and I-enantiomers of amphetamine or a pharmaceutically acceptable salt thereof are present in weight ratio of about 3.2 to about 1.

In a further aspect, amphetamine or a pharmaceutically acceptable salt, enantiomer and cation exchange resin are present in weight ratio of about 1 to about 3.

In a further aspect, the oral pharmaceutical composition does not need an uncoated amphetamine-ion exchange resin complex particles and amphetamine particles which are not complexed with an ion exchange resin, to provide extended release up to about 24 hours.

In a further aspect, ethyl acrylate and methyl methacrylate copolymer is present in a matrix with the drug-cation exchange resin complex in an amount of about 3% to about 30% by weight, based on the weight of said drug-cation exchange resin complex.

In a further aspect, the ethyl acrylate and methyl methacrylate copolymer is present in an extended-release coating over the drug-cation exchange resin complex-matrix in an amount of about 50% to about 90% by weight, based on the weight of said drug-cation exchange resin complex-matrix.

In a further aspect, the ethyl acrylate and methyl methacrylate copolymer in matrix and coating is Eudragit NE.

In a further aspect, the extended-release coating comprises 30% to 70% by weight of the uncoated complex-matrix.

The extended-release coated drug-cation exchange resin complex particles preferably provide about 8 hours, about 12 hours, about 18 hours, or about 24 hours release profile.

In another aspect, the present invention provides a method of manufacture of drug-ion exchange resin complexes that provide flexibility, higher drug binding efficiency, and drug loading and processing benefits to produce such complexes.

In a further aspect, the present invention provides an orally ingestible aqueous liquid suspension comprising—
(a) a plurality of extended-release coated drug-cation exchange resin complex particles which comprise—
   (i) particulate matrix comprising matrix of amphetamine-cation exchange resin complex and ethyl acrylate and methyl methacrylate copolymer,
   (ii) an extended-release coating over the drug-cation exchange resin complex-matrix particles comprising ethyl acrylate and methyl methacrylate copolymer, hydroxypropyl methylcellulose, a plasticizer and a glidant; and
(b) a pharmaceutically acceptable aqueous suspension base, wherein said extended-release coated drug-cation exchange resin complex-matrix particles are suspended in said base.

In a further aspect, the orally ingestible aqueous liquid suspension comprises the particulate matrix which comprises about 0.1 to about 10% of ethyl acrylate and methyl methacrylate copolymer by total weight of the extended-release coated drug-cation exchange resin complex particles.

In a further aspect, the orally ingestible aqueous liquid suspension comprises an extended-release coating which comprises about 7 to about 15% of ethyl acrylate and methyl methacrylate copolymer by total weight of the extended-release coated drug-cation exchange resin complex-matrix particles.

In another aspect, the present invention provides a method of treating or preventing ADHD comprising administering to a subject in need thereof the oral pharmaceutical composition or the aqueous liquid suspension as substantially described throughout the specification.

Still other aspects and advantages of the invention will be apparent from the following detailed description of the invention.

### DETAILED DESCRIPTION OF THE INVENTION

The present invention provides a coated drug-ion exchange resin composition for further use in formulation with conventional pharmaceutically acceptable components to provide ingestible compositions. The finished dose compositions may take the form of liquid preparations such as suspensions or solid preparations such as tablets, capsules, liquigels, powders, wafers, strips, etc.

The inventors have found that by using a polymer or copolymer in the diffusion barrier coating of drug-ion exchange resin which is the same as that used as impregnating (solvating) agent for preparing drug-ion exchange resin complex-matrix particles, a simple, improved and extended-release coated drug-ion exchange resin composition can be obtained.

The inventors have found that by using the same polymer or copolymer in the matrix and the coating, the issue of swelling and fracturing of the resin particle in biological solutions can be addressed. Additionally, coatability of the coating over the drug-ion exchange resin complex-matrix particles was found to have improved as a result of such selection. The specific extended-release coating need not be based upon the use of organic solvents to dissolve the coating solution and is also stable enough to maintain its film integrity and as a result to provide extended release of amphetamine in the gastrointestinal tract for a duration of up to about 24 hours.

Such oral pharmaceutical composition may provide extended release up to about 24 hours without the necessity of uncoated particulate amphetamine-ion exchange resin complex particles and amphetamine particles which are not complexed with an ion exchange resin.

A further desirable advantage, previously reported when using ion exchange resins, is to provide a reduction of undesirable tastes sometimes associated with an orally ingestible formulation, where unbearable or bad taste of the active drug may be a drawback to the recommended drug ingestion regimen.

The term “about” means the referenced numeric indication plus or minus 10% of that referenced numeric indication.

The term “matrix” as used herein throughout the specification denotes that the drug or drug-ion exchange resin complex, one or more polymers and/or copolymers are dispersed with one or more pharmaceutically acceptable excipients either homogeneously or heterogeneously. For example, in the homogeneous matrix system the drug, polymer/copolymers and excipients are distributed uniformly over the particle, while in the heterogeneous matrix system the drug, polymer/copolymers and excipients are non-uniformly distributed over the entire core.

The term “extended-release” refers to compositions of the invention which are characterized by having a drug release from a drug-ion exchange complex of the invention over a period of at least about 8 hours, at least about 12 hours, and preferably up to about 24 hours.

The term “extended-release coating” refers to a pH dependent, pH independent substance or mixture thereof that will act as a barrier to control the diffusion of the drug from its core complex into the gastrointestinal fluids.

Amphetamine, its pharmaceutically acceptable salts and enantiomers contemplated to be within the scope of the present invention include amphetamine base, all chemical and chiral derivatives or enantiomers (-d and -I) and salts thereof indicated for the treatment of attention deficit hyperactivity disorder (ADHD).

In a preferred embodiment, the composition comprise d- and I-enantiomers of amphetamine in weight ratio of about 3.2 to about 1.

In an embodiment, the drug release pattern from the composition of the present invention is controlled or modified by combining the drug and resin to form the drug-ion exchange resin complex-matrix prior to the application of the coating. The preferred polymer or copolymer useful in the extended-release coating is most preferably the same polymer or copolymer that is used as the impregnating (solvating) agent for the matrix. The extended-release coating system could be further customized by the incorporation of individual or a combination of hydrophilic or lipophilic plasticizers with a dispersion or suspension containing the barrier coating polymer. Such plasticizers include, e.g., propylene glycol, polyethylene glycol, triacetin, triethyl citrate, dibutyl sebacate, vegetable oil, lipids, etc.

Due to the extended drug release of up to about 24 hours, the compositions of the present invention have concomitant advantages: instead of taking two or three dosages per day, one may take a once-a-day dose that would provide more consistent supply (release) of the drug that otherwise may have to be taken multiple times a day. This is especially beneficial in the case of small children, elderly people, or others, who have difficulty swallowing larger solid dosage forms such as tablets or capsules.

The coated drug-ion exchange resins of the present invention are formulated into finished ingestible dosage forms such as a liquid suspension or a fast disintegrating tablet that need not be swallowed. It has also been observed that for use in liquid compositions, the coating of the present invention for the drug-ion exchange resin complex when formulated into a liquid suspension does not swell and fracture in the biological fluids so the particles retain their geometry. Advantageously, the particles of the invention may not produce undesirable agglomerations and colour migration in the liquid known in the prior art with use of EUDRAGIT grade polymers in the presence of a colorant which is desirably used in medicines to be taken by children. Therefore, such extended release compositions may enhance compliance.

### Ion Exchange Resin—

Ion-exchange resins suitable for use in these preparations are water-insoluble and comprise a preferably pharmacologically inert organic and/or inorganic matrix containing functional groups that are ionic or capable of being ionized under the appropriate conditions of pH. The organic matrix may be synthetic (e.g., polymers or copolymers of acrylic acid, methacrylic acid, sulfonated styrene, sulfonated divinylbenzene), or partially synthetic (e.g. modified cellulose and dextrans). The inorganic matrix preferably comprises silica gel modified by the addition of ionic groups. Covalently bound ionic groups may be strongly acidic (e.g., sulfonic acid, phosphoric acid), weakly acidic (e.g., carboxylic acid), strongly basic (e.g., primary amine), weakly basic (e.g. quaternary ammonium), or a combination of acidic and basic groups. In general, the types of ion exchangers suitable for use in ion-exchange chromatography and for such applications as deionization of water are suitable for use in the controlled release of drug preparations. Such ion-exchangers are described by H. F. Walton in “Principles of Ion Exchange” (pp: 312-343) and “Techniques and Applications of Ion-Exchange Chromatography” (pp: 344-361) in Chromatography. (E. Heftmann, editor), van Nostrand Reinhold Company, New York (1975). Ion exchange resins that can be used in the present invention have exchange capacities of about 6 milliEquivalents (mEq)/gram and preferably about 5.5 mEq/gram or below.

Typically the size of the ion-exchange particles is from about 5 microns to about 750 microns, preferably the particle size is within the range of about 40 microns to about 250 microns for liquid dosage forms although particles up to about 1,000 micron can be used for solid dosage forms, e.g., tablets and capsules.

Two of the preferred resins of this invention are Amberlite IRP-69 and Dow XYS-40010.00. Both are sulfonated polymers composed of polystyrene cross-linked with about 8% of divinylbenzene, with an ion-exchange capacity of about 4.5 to 5.5 meq/g of dry resin (H^{±} form). Their essential difference is in physical form. Amberlite IRP-69 consists of irregularly shaped particles with a size range of about 5 microns to about 149 microns produced by milling the parent large size spheres of Amberlite IRP-120. The Dow XYS-40010.00 product consists of spherical particles with a size range of 45 microns to 150 microns.

Cation exchange resins, e.g., AMBERLITE IRP-69, are particularly well suited for use with drugs and other molecules having a cationic functionality, including amphetamine.

In an embodiment, cation exchange resin, preferably sulfonated copolymer of a polystyrene crosslinked with divinylbenzyl and most preferably AMBERLITE IRP-69 is the choice of ion exchange resin for preparing extended-release compositions of amphetamine.

### Drug-Ion Exchange Resin Complexes—

Binding of drug to resin can be accomplished according to four general reactions. In the case of a basic drug, these are: (a) resin (Na-form) plus drug (salt form); (b) resin (Na-form) plus drug (as free base); (c) resin (H-form) plus drug (salt form); and (d) resin (H-form plus drug (as free base). All of these reactions except (d) have cationic by-products and these by-products, by competing with the cationic drug for binding sites on the resin, reduce the amount of drug bound at equilibrium. For basic drugs, stoichiometric binding of drug to resin is accomplished only through reaction (d).

Four analogous binding reactions can be carried out for binding an acidic drug to an anion exchange resin. These are: (a) resin (Cl-form) plus drug (salt form); (b) resin (Cl-form) plus drug (as free acid); (c) resin (as free base) plus drug (salt form); and (d) resin (as free base) plus drug (as free acid). All of these reactions except (d) have ionic by-products and the anions generated when the reactions occur compete with the anionic drug for binding sites on the resin with the result that reduced levels of drug are bound at equilibrium. For acidic drugs, stoichiometric binding of drug to resin is accomplished only through reaction (d).

In an embodiment, drug and ion exchange resin in the complex are present in weight ratio of about 1 to about 3.

Typically the drug-ion exchange resin complex thus formed is collected by filtration and washed with appropriate solvents to remove any unbound drug or by-products. The complexes can be air-dried in trays, in a fluid bed dryer, or other suitable dryer, at room temperature or at elevated temperature.

Matrix system—

The drug release rate from the compositions of the present invention is further prolonged or modified by treating the drug-ion exchange resin complex prior to the application of the coating described herein, with one or more acrylic based polymers or copolymers. Advantageously, these acrylic based polymers or copolymers does not form a separate layer on the drug-ion exchange resin complex, but forms a matrix therewith.

Most preferred copolymer for use in the matrix is acrylic based copolymer (e.g., represented by the EUDRAGIT family of acrylic resins). Examples of suitable acrylic polymers from the EUDRAGIT family may include, e.g., a copolymer comprising ethyl acrylate and methyl methacrylate (e.g., EUDRAGIT NE-30D), or EUDRAGIT RS, RL30D, RL100, or NE, which are largely pH-independent polymers; although less desirable, certain pH-dependent members of the EUDRAGIT polymer family, e.g., the L, S, and E, polymers may be selected. In a preferred embodiment, ethyl acrylate and methyl methacrylate copolymer in coating is a pH-independent copolymer, most preferably EUDRAGIT NE-30D.

However, additional polymers such as polyvinyl acetate polymer or a mixture of polymers containing same (e.g., KOLLICOAT SR 30D), cellulose acetates, ethylcellulose polymers (e.g., AQUACOAT™ ECD-30 or SURELEASE™), cellulose phthalate may be used to customize the desired release profile.

The quantity of polymer/copolymer that is used in the matrix typically ranges from about 3% to about 30% or more by weight of the uncoated drug-ion exchange resin particles. More preferably the release retardant, if used, is in the range from about 5% to about 20% and most preferably in the range of about 10% to about 15% by weight of the uncoated drug-ion exchange resin particles, depending on the nature of the drug-ion exchange resin complex and the desired release profile of the drug.

These polymers/copolymers can be added during the formation of the drug-ion exchange resin complex either in the beginning, during the middle, or after substantial amount of complex formation has taken place. In the more preferred embodiment, the retardant is added after the formation of drug-ion exchange resin complex. Upon admixing, the drug-ion exchange resin complex particles with the polymer/copolymer, the mixture is dried and milled appropriately. In some cases, the milling may be carried out before the complete drying of the complex and then again further drying followed by milling to obtain the desired complex characteristics.

### Coating System—

The most preferred polymer or copolymer for use in the coating is acrylic based polymer or copolymer (e.g., represented by the EUDRAGIT family of acrylic resins). Examples of suitable acrylic polymers from the EUDRAGIT family may include, e.g., a copolymer comprising ethyl acrylate and methyl methacrylate (e.g., EUDRAGIT NE-30D), or EUDRAGIT RS, RL30D, RL100, or NE, which are largely pH-independent polymers; although less desirable, certain pH-dependent members of the EUDRAGIT polymer family, e.g., the L, S, and E, polymers may be selected.

In a preferred embodiment, ethyl acrylate and methyl methacrylate copolymer in coating is a pH-independent copolymer, most preferably EUDRAGIT NE.

In one embodiment, the coating layer is about 5% to about 200%, by weight, of the uncoated drug-ion exchange resin complex. In another embodiment, the barrier coating layer is about 30% to about 70% by weight of the uncoated drug-ion exchange resin complex, about 40% to about 60% by weight of the uncoated complex.

The coating may comprise suitable water soluble and/or water insoluble plasticizers. Examples of suitable plasticizers include, e.g., dibutyl sebacate, propylene glycol, polyethylene glycol, polyvinyl alcohol, triethyl citrate, acetyl triethyl citrate, acetyl tributyl citrate, tributyl citrate, triacetin, and Soluphor P, and mixtures thereof.

It may be desirable to incorporate a water-soluble substance as pore formers, such as methylcellulose, hydroxypropyl methylcellulose to alter the permeability of the coating.

The coating composition may include conventional additives, such as plasticizers, pigments, colorants, stabilizing agents, glidants, etc. A plasticizer is normally present to reduce the fragility of the coating, and will generally represent about 10% to 50% by weight relative to the dry weight of the polymer. Examples of typical plasticizers are, but not limited to, polyethylene glycol, propylene glycol, triacetin, dimethyl phthalate, diethyl phthalate, dibutyl phthalate, dibutyl sebacate, triethyl citrate, tributyl citrate, triethyl acetyl citrate, castor oil and acetylated monoglycerides. A stabilizing agent may be used to stabilize particles in the dispersion. Typical stabilizing agents are non-ionic emulsifiers such as sorbitan esters, polysorbates and polyvinylpyrrolidone. Glidants are recommended to reduce sticking effects during film formation and drying, and will generally represent approximately 25% to 100% by weight of the polymer weight in the coating solution. One preferred glidant is talc. Other glidants such as magnesium stearate and glycerol monostearates may also be used. Pigments such as titanium dioxide may also be used. Small quantities of an anti-foaming agent, such as a silicone (e.g., simethicone), may also be added to the coating composition.

The release rate of the present coatings of the invention which are designed to provide finished dosage orally ingestible pharmaceutical compositions such as liquid suspension, tablets, etc. are tailored to provide the desired drug release profile over a period of about 8 to 24 hours, and preferably 12 to 24 hours.

### Finished Dosage Form—

The coated drug-ion exchange resin complex particles of the invention may be formulated for delivery by any suitable route including, e.g., orally, topically, intraperitoneally, transdermally, sublingually, intramuscularly, rectally, transbuccally, intranasally, liposomally, via inhalation, vaginally, intraoccularly, via local delivery (for example, by catheter or stent), subcutaneously, intraadiposally, intraarticularly, or intrathecally. Preferably, the complex is formulated for oral delivery.

The drug-ion exchange resin composition thus prepared may be stored for future use or promptly formulated with conventional pharmaceutically acceptable carriers to prepare finished ingestible compositions for delivery orally, nasogastric tube, or via other means. The compositions according to this invention may, for example, take the form of liquid preparations such as suspensions, or solid preparations such as capsules, tablets, caplets, sublinguals, powders, wafers, strips, gels, including liquigels, etc. In one embodiment, a tablet of the invention is formulated as an orally disintegrating tablet. Such orally dissolving tablets may disintegrate in the mouth in less than about 60 seconds.

The drug-ion exchange resin coated compositions may be formulated using conventional pharmaceutically acceptable carriers or excipients and well established techniques. Without being limited thereto, such conventional carriers or excipients include diluents, binders and adhesives (i.e., cellulose derivatives and acrylic derivatives), lubricants (i.e., magnesium or calcium stearate, or vegetable oils, polyethylene glycols, talc, sodium lauryl sulfate, polyoxy ethylene monostearate), thickeners, solubilizers, humectants, disintegrants, colorants, flavorings, stabilizing agents, sweeteners, and miscellaneous materials such as buffers and adsorbents in order to prepare a particular pharmaceutical composition. The stabilizing agents may include preservatives and anti-oxidants, amongst other components which will be readily apparent to one of ordinary skill in the art.

Suitable thickeners include, e.g., tragacanth; xanthan gum; bentonite; starch; acacia and lower alkyl ethers of cellulose (including the hydroxy and carboxy derivatives of the cellulose ethers). Examples of cellulose include, e.g., hydroxypropyl cellulose, hydroxypropyl methyl cellulose, sodium carboxy methylcellulose, microcrystalline cellulose (MCC), and MCC with sodium carboxyl methyl cellulose. In one embodiment, tragacanth is used and incorporated in an amount of from about 0.1 to about 1.0% weight per volume (w/v) of the composition, and more preferably about 0.5% w/v of the composition. Xanthan gum is used in the amount of from about 0.025 to about 0.5% w/v and preferably about 0.25% w/v.

The extended-release ion exchange resin compositions may include a humectant composition to give the liquid greater viscosity and stability. Suitable humectants useful in the finished formulations include glycerin, polyethylene glycol, propylene glycol and mixtures thereof.

The oral liquid compositions of the present invention may also comprise one or more surfactants in amounts of up to about 5.0% w/v and preferably from about 0.02 to about 3.0% w/v of the total formulation. The surfactants useful in the preparation of the finished compositions of the present invention are generally organic materials which aid in the stabilization and dispersion of the ingredients in aqueous systems for a suitable homogenous composition. Preferably, the surfactants of choice are non-ionic surfactants such as poly(oxyethylene)(20) sorbitan monooleate and sorbitan monooleate. These are commercially known as TWEENS and SPANS and are produced in a wide variety of structures and molecular weights.

Whereas any one of a number of surfactants may be used, preferably a compound from the group comprising polysorbate copolymers (sorbitan-mono-9-octadecenoate-poly(oxy-1,2-ethanediyl)) is employed. This compound is also added functions to keep any flavors and sweeteners homogeneously dissolved and dispersed in solution.

Suitable polysorbates include polysorbate 20, polysorbate 40, polysorbate 80 and mixtures thereof. Most preferably, polysorbate 80 is employed. The surfactant component will comprise from about 0.01 to about 2.0% w/v of the total composition and preferably will comprise about 0.1% w/v of the total weight of the composition.

Aqueous suspensions may be obtained by dispersing the drug-ion exchange resin compositions in a suitable aqueous vehicle, optionally with the addition of suitable viscosity enhancing agent(s) (e.g., cellulose derivatives, xanthan gum, etc). Non-aqueous suspensions may be obtained by dispersing the foregoing compositions in a suitable non-aqueous based vehicle, optionally with the addition of suitable viscosity enhancing agent(s) (e.g., hydrogenated edible fats, aluminum state, etc.). Suitable non-aqueous vehicles include, for example, almond oil, arachis oil, soybean oil or soybean oil or fractionated vegetable oils such as fractionated coconut oil.

The aqueous suspension further may include one or more pharmaceutically acceptable preservatives, sweeteners, flavoring agents, colourants and wetting agents.

### EXAMPLE 1

### Amphetamine Extended-Release Liquid Oral Suspension

TABLE 1

| Step | Stage | Name of the material | Mg/unit |
|---|---|---|---|
| | | Amphetamine salts | 4.0 |
| | | Amberlite IRP-69 | 7.5 |
| | | Purified water | 80.0 |
| 2 | Partial Drying | Amphetamine-Resin complex | 10.0 |
| 3 | Impregnation | Ethyl Acrylate and Methyl Methacrylate | 1.0 |
| | | Copolymer (Eudragit NE 30D- | |
| | | 10% Solid) | |
| 4 | Complete | Amphetamine-Resin complex-matrix | 11.0 |
| | Drying | | |
| | | Ethyl Acrylate and Methyl Methacrylate | 4.5 |
| | | Copolymer (Eudragit NE 30D-40% Solid) | |
| | | Hydroxypropyl methylcellulose, 3 cps | 0.45 |
| | | (10%) | |
| | | Polysorbate 80 (10%) | 0.45 |
| | | Talc | 4.5 |
| | | Purified water | 60.0 |
| | | Extended-release coated Amphetamine- | 20.9 |
| | | Resin complex-matrix particles | |

| | |
|---|---|
| Total polymer weight gain | 50% |

### Procedure:

(1) Complexation: 3 parts of Amberlite IRP-69 were dispersed in 1 part of mixed amphetamine salts, 5 parts of purified water, and the dispersion was stirred for 24 hours. The filter was decanted after 24 hours and the drug-resin complex (resonate) was washed adequately twice.
(2) Partial Drying: Washed resonate was dried at 60° C. to LOD of 20% moisture in a fluid bed dryer or tray dryer.
(3) Impregnation: 10% dry polymer equivalent liquid Eudragit NE 30D was added to the partially dried resonate while mixing in a planery mixer for impregnation by adsorption and the material was dried further.
(4) Complete Drying: The impregnated resonate was dried further at 60° C. to get LOD of 5-10% in fluid bed dryer or tray dryer. The dried impregnated resonate complex was milled and pass through 100 mesh screen.
(5) ER Coating: Extended release coating solution prepared with 40% dry polymer equivalent liquid Eudragit NE 30D, 10% HPMC (as pore former) was sprayed over the milled and screened impregnated resonate complex to get 40% weight gain in the fluid bed coater followed by drying and curing. The SR coated resonate were through 80 mesh screen and tested for potency.
(6) ER Suspension: The passed SR coated resonate complex particles were mixed in placebo liquid suspension base to produce a SR suspension containing 2.5 mg of amphetamine base per mL.

What is claimed is:

1. An oral pharmaceutical composition comprising an extended-release coating over one or more drug-cation exchange resin complex particles, said particles comprising amphetamine, a pharmaceutically acceptable salt, enantiomer, or a combination thereof bound to a cation exchange resin, wherein:
(a) said extended-release coated drug-cation exchange resin complex particles comprise ethyl acrylate and methyl methacrylate copolymer in a matrix with the one or more drug-cation exchange resin complex particles, and
(b) the extended-release coating over the drug-cation exchange resin complex-matrix particles comprise ethyl acrylate and methyl methacrylate copolymer.

2. The oral pharmaceutical composition of claim 1, wherein the cation exchange resin is a sulfonated copolymer of a polystyrene crosslinked with divinylbenzyl.

3. The oral pharmaceutical composition of claim 1, wherein the ethyl acrylate and methyl methacrylate copolymer in the matrix and the coating is Eudragit NE.

4. The oral pharmaceutical composition of claim 1, wherein the drug-cation exchange resin complex particles have a particle size in the range of about 40 to about 250 microns.

5. The oral pharmaceutical composition of claim 1, wherein the composition comprises a mixture of d- and I-enantiomer of amphetamine or a pharmaceutically acceptable salt thereof.

6. The oral pharmaceutical composition of claim 5, wherein d- and I-enantiomers of amphetamine are present in a weight ratio of about 3.2 to about 1.

7. The oral pharmaceutical composition of claim 1, wherein amphetamine, a pharmaceutically acceptable salt, enantiomer, or combination thereof and cation exchange resin are present in weight ratio of about 1 to about 3.

8. The oral pharmaceutical composition of claim 1, wherein the composition is devoid of uncoated amphetamine-ion exchange resin complex particles, and amphetamine particles which are not complexed with an ion exchange resin.

9. The oral pharmaceutical composition of claim 1, wherein the ethyl acrylate and methyl methacrylate copolymer in a matrix with the drug-ion exchange resin complex is present in an amount of about 3% to about 30% by weight, based on the weight of said drug-cation exchange resin complex.

10. The oral pharmaceutical composition of claim 1, wherein the ethyl acrylate and methyl methacrylate copolymer in the extended-release coating over the drug-ion exchange resin complex-matrix is present in an amount of about 50% to about 90% by weight, based on the weight of said drug-cation exchange resin complex-matrix.

11. The oral pharmaceutical composition of claim 1, wherein the extended-release coating comprises 30% to 70% by weight of the uncoated complex-matrix.

12. The extended-release coated drug-cation exchange resin complex particles of claim 1 that provides at least about 8 hour release profile.

13. An orally ingestible aqueous liquid suspension comprising the extended-release coated drug-cation exchange resin complex particles of claim 1 suspended in a pharmaceutically acceptable aqueous suspension base.

14. An orally ingestible aqueous liquid suspension comprising:
(a) A plurality of extended-release coated drug-cation exchange resin complex particles which comprise:
(i) a particulate matrix comprising matrix of amphetamine-cation exchange resin complex and ethyl acrylate and methyl methacrylate copolymer,
(ii) an extended-release coating over the drug-cation exchange resin complex-matrix particles comprising ethyl acrylate and methyl methacrylate copolymer, hydroxypropyl methylcellulose, a plasticizer and a glidant; and
(b) a pharmaceutically acceptable aqueous suspension base, wherein said extended-release coated drug-cation exchange resin complex-matrix particles are suspended in said base.

15. The orally ingestible aqueous liquid suspension of claim 14, wherein the particulate matrix comprises about 0.1 to about 10% of ethyl acrylate and methyl methacrylate copolymer by total weight of extended-release coated drug-cation exchange resin complex particles.

16. The orally ingestible aqueous liquid suspension of claim 14, wherein the extended-release coating comprises about 7 to about 15% of ethyl acrylate and methyl methacrylate copolymer by total weight of extended-release coated drug-cation exchange resin complex-matrix particles.

17. The orally ingestible aqueous liquid suspension of claim 14, wherein the suspension is devoid of uncoated amphetamine-ion exchange resin complex particles, and amphetamine particles which are not complexed with an ion exchange resin.

18. The composition of claim 14, wherein the extended-release coating comprises 30% to 70% by weight of the uncoated complex.

19. A method for treating or preventing ADHD comprising administering the oral pharmaceutical composition of claim 1 to a subject in need thereof.
